(19) United States
(12) Patent Application Publication
(10) Pub. No.: US 2007/0067873 A1
(43) Pub. Date: Mar. 22, 2007

(54) Modified ppase expression in sugar beet

(76) Inventors: Steffen Greiner, Offenbach (DE); Karsten Harms, Grunstadt (DE); Markwart Kunz, Worms (DE); Mohammad Munir, Kindenheim (DE); Thomas Rausch, Kindenheim (DE); Markus Schirmer, Heidelberg (DE)

Correspondence Address: OSTROLENK FABER GERB & SOFFEN, 1180 AVENUE OF THE AMERICAS, NEW YORK, NY, 100368403, US

(21) Appl. No.: 10/549,997

(22) PCT Filed: Feb. 14, 2004
(86) PCT No.: PCT/EP04/01405

§ 371 Date: Dec. 16, 2005

(30) Foreign Application Priority Data
Mar. 20, 2003 (DE) 103 13 795.5

(51) Int. Cl. A01H 1/00; C12N 15/82; C12N 5/04

(52) U.S. Cl. 800/284; 435/419; 435/468

(57) ABSTRACT

The present invention relates to a process and means for producing an improved sugar beet, in particular a sugar beet which exhibits an increased content of sucrose, a reduced rate of sucrose breakdown during storage and an improved growth. The invention also relates to the use of at least two gene constructs for generating such a plant and to nucleotide sequences which are employed in this connection.

The present invention relates to a process and means for producing an improved sugar beet, in particular a sugar beet which exhibits an increased sucrose content in its storage organ, a reduced breakdown of sucrose during storage and an increased growth of the beet. In particular, the invention relates to the use of at least two gene constructs for generating such a plant and to nucleotide sequences which are employed in this connection.

During the storage of sugar beet (Beta vulgaris), that is during the period between harvesting and further processing, in particular sugar extraction, substantial losses of sucrose occur as a result of sucrose being broken down in the storage organs. This breakdown of sucrose also takes place, for the purpose of sustaining a maintenance metabolism in the beet body, after the beet has stopped growing. It is mainly the sucrose which has accumulated in the beet body which is broken down while the beet are being stored. While the breakdown of the sucrose is on the one hand dependent on a variety of environmental factors, it is also dependent on the harvesting process. It is also coupled to a decrease in the quality of the sugar beet since, as a result, the proportion of reducing sugars such as fructose or glucose in the beet body increases (Burba, M., Zeitschrift für die Zuckerindustrie [The Sugar Industry Journal] 26 (1976), 647-658).

In the wound region of topped harvested beet, for example, the breakdown of sucrose is first and foremost mediated by enzymic hydrolysis brought about by a wound-induced invertase which is primarily located in the vacuoles of the beet cells. Vacuolar and/or cell wall-bound invertase isoforms are also induced when beet tissue is wounded de novo (Rosenkranz, H. et al., J. Exp. Bod. 52 (2001), 2381-2385). This process can be countered by expressing an invertase inhibitor (WO 98/04722) or by expressing an antisense RNA construct or a dsRNA construct for vacuolar invertase (WO 02/50109). However, this only partially prevents sucrose being broken down in the beet body. This is principally because sucrose is broken down to a significant extent, by way of sucrose synthase acting in reverse, UGPase and PFP, in the remainder of the beet body, that is outside the wound region, mainly as a consequence of the anaerobic conditions which prevail in this area. Cytosolic inorganic pyrophosphate (PPᵢ) is required for the enzymic activity of the UGPase (uridine diphosphoglucose pyrophosphorylase) and the PFP (pyrophosphate:fructose 6-phosphate phosphotransferase) in this breakdown pathway (Stitt, M., Bot. Acta 111 (1998), 167-175).

It is known that dissimilating enzyme reactions, which are dependent on cytosolic pyrophosphate as the energy supplier, take place in the plant cell, principally under anaerobic conditions, in addition to ATP-dependent metabolic processes. Accordingly, essentially two different pathways for breaking down sucrose exist in the plant cell (Stitt, M., loc. cit.):
1) Hydrolysis of the sucrose into fructose and glucose by invertase, with the hexose, which is phosphorylated by hexokinase and fructokinase in the presence of ATP, being converted by phosphofructokinase (PFK), likewise with ATP being consumed, into fructose 1,6-bisphosphate.
2) The breakdown of sucrose by sucrose synthase into UDP-glucose and fructose, with subsequent conversion of the UDP-glucose into hexose phosphate by UGPase in the presence of pyrophosphate and conversion of the hexose phosphate into fructose 1,6-bisphosphate by PFP, likewise in the presence of pyrophosphate.

The second, PPᵢ-dependant breakdown pathway is even preferentially taken in the plant cell under anaerobic conditions which arise when the beet bodies are stored since this thereby conserves ATP reserves which would be consumed in the first of the pathways for breaking down sucrose. Since previously known measures for reducing the loss of sucrose principally relate to inhibiting the first breakdown pathway (for example invertase inhibition), which, except in wound regions, is of little relevance for the loss of sucrose in stored beets, there is currently no satisfactory solution to the problem of sucrose losses which are occasioned by storage. Other known measures consist of a general reduction in enzymic activity which is achieved by storing at low temperatures, for example less than 12° C., while at the same time maintaining a high atmospheric humidity.

In addition, there is the need to make available plants, in particular beet plants, which already exhibit an increased content of sucrose in their storage organs, or beet plants which, as a result of increased growth as a consequence of a longer period of meristem activity, also form a larger beet body and thus store more sucrose.

Meristematic tissues exhibit an intensive pyrophosphate metabolism. Centrally important synthetic activities in the meristems, such as cell wall synthesis, protein synthesis and nucleic acid synthesis, form pyrophosphate as a reaction product, which means that its cleavage promotes the enzyme reactions concerned. For this reason, the control of the pyrophosphate pool in the cytoplasm and nucleus by enzyme reactions which cleave or consume pyrophosphate constitutes an important mechanism for influencing meristematic activity. Vacuolar H⁺-pyrophosphatases and soluble pyrophosphatases are involved in this connection, in addition to enzyme reactions which use pyrophosphate as cosubstrate (PFP and UGPase, see above).

The object of the present invention is therefore to provide a system which essentially further reduces sucrose losses in plants, in particular beet plants, and also leads to plants which exhibit an increase in the content of sucrose and/or an increase in the size of the beet body.

According to the invention, this object is achieved by providing a process for producing a transgenic plant, in particular a beet plant, preferably sugar beet (Beta vulgaris) which exhibits an increased content of sucrose, and preferably a decreased breakdown of sucrose, during storage, as claimed in claim 1. The object is also achieved, in accordance with the invention, by providing a transgenic plant which can be obtained by means of this process and which exhibits an increased content of sucrose and, in particular, a decreased breakdown of sucrose during storage. The object is also achieved, in accordance with the invention, by providing at least one nucleic acid molecule encoding a protein having the biological activity of a Beta vulgaris soluble pyrophosphatase, in particular a cytosolic pyrophosphatase (C-PPase), preferably the same pyrophosphatase whose compartmentation is altered by inserting at least one nuclear localization sequence (NLS), as well as by providing at least one nucleic acid molecule which encodes a promoter of a Beta vulgaris vacuolar pyrophosphatase (V-PPase).

The process according to the invention for producing a transgenic beet plant having an increased content of sucrose comprises
a) transforming at least one beet cell with at least two transgenes, with the first transgene encoding a vacuolar pyrophosphatase (V-PPase), in particular from Beta vulgaris, and the second transgene encoding a cytosolic or nucleus-located soluble pyro-phosphatase (C-PPase), in particular from Beta vulgaris, and, following that,
b) culturing and regenerating the at least one beet cell which has been transformed in this way under conditions which lead to the partial, preferably complete, regeneration of a transgenic beet plant having an increased content of sucrose, with
c) a transgenic, regenerated beet plant having an increased content of sucrose in the beet then being obtained, which beet plant exhibits an increased sucrose content in the beet, preferably a decreased breakdown of sucrose during storage, and/or, preferably, a beet body which is increased in size due to an increase in meristem activity.

The inventors have found, surprisingly, that simultaneously expressing a nucleic acid molecule which is provided as the first transgene and which encodes a V-PPase, in particular from Beta vulgaris, preferably a V-PPase cDNA, and a nucleic acid molecule which is provided as the second transgene and which encodes a C-PPase, in particular from Beta vulgaris, preferably a C-PPase cDNA, in the transgenic cell of a beet plant restricts the flux of sucrose from the vacuole, increases the transport of sucrose into the vacuole and minimizes the cytosolic breakdown of the sucrose on the PPᵢ-dependent pathway. The decrease in the availability of vacuolar sucrose in the cytosol in this connection is primarily to be attributed to the increase in the activity of the ΔpH-dependent sucrose transport of sucrose into the vacuole by way of the tonoplast membrane. The pH gradient which is required for the sucrose transport is to a high degree dependent on the activity of the membrane-located V-PPase. This latter exhibits a high activity (K_{M}<10 μmol/l) even in the presence of a low concentration of the substrate pyrophosphate, whereas the affinity of soluble PPases is markedly lower (K_{M}>100 pmol/l). Surprisingly, the process according to the invention makes it possible to obtain a transgenic plant cell, in particular a transgenic plant, in which the accumulation of sucrose is increased.

The content of pyrophosphate in the plant cell is reduced by the expression, in particular the overexpression, which is mediated in accordance with the invention, of transgenic cytosolic or nucleus-located pyrophophatase and/or transgenic vacuolar pyrophosphatase. In this connection, particular preference is given, in accordance with the invention, to the expression, in particular overexpression, which is mediated in accordance with the invention, of transgenic cytosolic or nucleus-located pyrophosphatase together with, preferably at the same time as, the expression, in particular overexpression, which is mediated in accordance with the invention, of transgenic vacuolar pyrophosphatase. On the One hand, this thereby crucially reduces the pyrophosphate-dependent breakdown of sucrose; on the other hand, the increased breakdown of pyrophosphate in the cytosol and cell nucleus also promotes a variety of synthetic activities in the meristems of the plant, with this in turn having a growth-increasing effect such that beet bodies which are increased in size are obtained. Advantageously, the increase in the activity of the V-PPase increases the sucrose content in the vacuole, significantly reduces the breakdown of sucrose in the cytosol and increases the activity of the meristems, in particular those which are located at the periphery of the growing beet body.

A transgenic plant which can be obtained in this way exhibits an increase in growth as well as, in particular, an increase in sucrose content, in particular already at the time of harvesting. The storage-associated breakdown of sucrose in the plant is reduced and the transgenic plant which can be obtained in this way is more stable during storage.

In connection with the present invention, an “increased content of sucrose” is understood as being a content of sucrose, principally in the storage tissue of plants, in particular beet, which is normally at least 5%, in particular at least 10%, preferably at least 20%, particularly preferably at least 30%, greater than the average content of sucrose in corresponding tissues of comparable, known beets. In the last 20 years in Germany, the average sucrose content in the storage root of the sugar beet (Beta vulgaris) has been 17.14±0.56% by weight (see, e.g., Zuckerindustrie [Sugar Industry] 126 (2001) 2: p. 162). Preference is given to the average content of sucrose in the storage tissue of the beets which can be obtained in accordance with the invention being more than 18% by weight, in particular more than 21% by weight.

In connection with the present invention, an “increased meristem activity” or an “improved meristem growth” is understood as meaning an increase in the growth of the beet (based on the fresh weight) of normally at least 5%, preferably at least 10%, particularly preferably at least 19%, as compared with the average growth of comparable, known beets.

In connection with the present invention, a “transgene” is understood as meaning a gene which can, in the form of DNA or RNA, preferably cDNA, be transfected, that is transformed, into a eukaryotic cell, resulting in foreign genetic information, in particular, being introduced into the transfected eukaryotic cell. In this connection, a “gene” is understood as meaning at least one nucleotide sequence, that is one or more information-carrying segments of DNA molecules, which is under the operative control of at least one regulatory element and which, in particular, is protein-encoding. After the eukaryotic cell has been transfected, transgenes are present transiently, or else integrated into the genome of the transfected cell, as (a) nucleic acid molecule(s), with these latter not naturally occurring in this cell, or else they are integrated at a site in the genome of this cell at which they do not naturally occur, that is transgenes are located in a different genomic environment or are present in a copy number which is different from the natural copy number or are under the control of a different promoter.

According to the invention, the first transgene, which encodes a V-PPase, in particular from Beta vulgaris, preferably comprises at least one nucleic acid molecule, with the sequence of this nucleic acid molecule being selected from the group consisting of
a) a nucleotide sequence depicted in sequence ID No. 4, the sequence which is complementary thereto,
b) a nucleotide sequence which encodes the amino acid sequence depicted in sequence ID No. 5, and also its complementary nucleotide sequence, and
c) a modified nucleotide sequence, with a modified nucleic acid molecule of the modified nucleotide sequence hybridizing with the nucleic acid molecule having the nucleotide sequence in accordance with a) or b) and, in this connection, exhibiting a sequence identity of more than 80%, preferably more than 90%, 95% or 99%.

According to the invention, the second transgene, which encodes a C-PPase, in particular from Beta vulgaris, preferably comprises at least one nucleic acid molecule, with the sequence of this nucleic acid molecule being selected from the group consisting of
a) a nucleotide sequence depicted in sequence ID No. 1, the sequence which is complementary thereto,
b) a nucleotide sequence which encodes the amino acid sequence depicted in sequence ID No. 2, and also its complementary nucleotide sequence, and
c) a modified nucleotide sequence, with a modified nucleic acid molecule of the modified nucleotide sequence hybridizing with the nucleic acid molecule having the nucleotide sequence in accordance with a) or b) and, in this connection, exhibiting a sequence identity of more than 80%, preferably more than 90%, 95% or 99%.

In a preferred variant, the nucleotide sequence of the previously mentioned C-PPase nucleic acid molecule, which is preferred in accordance with the invention, also comprises at least one nuclear localization sequence.

In a preferred embodiment of the process according to the invention, the at least one first transgene is arranged on a vector. Preference is given, in accordance with the invention, to the at least one second transgene also being able to be arranged on a vector. Particular preference is given to both the first and the second transgene being arranged on a vector, in particular on the same vector. In a preferred version, the vector is present in isolated and purified form.

In a preferred embodiment of the process according to the invention, the at least one first transgene, encoding a V-PPase, and the at least one second transgene, encoding a C-PPase, are arranged together on a single vector, with, in particular, the first transgene being arranged in the 5′ to 3′ direction upstream of the second transgene. In an alternative variant, the second transgene is arranged in the 5′ to 3′ direction on the vector upstream of the first transgene. In another preferred embodiment, at least one first transgene is arranged on at least one first vector and at least one second transgene is arranged on at least one second vector which is different from the first vector.

In a particularly preferred embodiment, the first and second transgenes are transfected, that is transformed, simultaneously into at least one plant cell, in particular beet cell. The transformation is preferably carried out by means of ballistic injection, that is by means of biolistic transformation, in a manner which is known per se. In another variant, the transformation takes place by means of electro-transformation, preferably by means of electroporation, in a manner which is known per se. In another variant, the transformation is carried out using agrobacteria, preferably using, in particular, Agrobacterium tumefaciens or Agrobacterium rhizogenes as transformation means, in a manner which is known per se. In another variant, the transformation is carried out using viruses, in a manner which is known per se.

In connection with the present invention, “vectors” are understood as meaning, in particular, liposomes, cosmids, viruses, bacteriophages, shuttle vectors and other vectors which are customary in genetic engineering. “Vectors” are preferably understood as meaning plasmids. In a particularly preferred variant, the vector is the pBinAR vector (Hofgen and Willmitzer, 1990). These vectors preferably also possess at least one further functional unit which, in particular, brings about the stabilization and/or replication of the vector in the host organism, or contributes to this.

In a particularly preferred embodiment of the process according to the invention, use is made of vectors in which at least one nucleic acid molecule according to the invention is under the functional control of at least one regulatory element. According to the invention, the term “regulatory element” is understood as meaning elements which ensure the transcription and/or translation of nucleic acid molecules in prokaryotic and/or eukaryotic host cells such that a polypeptide or protein is expressed. Regulatory elements can be promoters, enhancers, silencers and/or transcription termination signals. Regulatory elements which are functionally linked to a nucleotide sequence according to the invention, in particular to the protein-encoding segments of this nucleotide sequence, can be nucleotide sequences which are derived from different organisms or different genes than the protein-encoding nucleotide sequence itself. In a preferred variant, the vector which is preferably employed in accordance with the invention possesses at least one further regulatory element, in particular at least one intrans enhancer.

The vectors which are used are preferably equipped for overexpressing the first or second transgene or both transgenes. This is achieved, in particular, by the at least one first transgene and/or the at least one second transgene being operatively linked, on the vector, to at least one promoter. Particular preference is given, in accordance with the invention, to the promoter being a tissue-specific promoter, a promoter which is constitutively expressing (=constitutive) or an inducible promoter. Preference is given, in accordance with the invention, to the promoter also being a storage-specific promoter. In a particularly preferred variant, the promoter on the above-mentioned vector possesses a combination of the properties of the above-mentioned promoters.

In a particularly preferred embodiment, the at least one promoter is a promoter from a beet plant, in particular from Beta vulgaris. This is preferably a promoter of the vacuolar pyrophosphatase (V-PPase promoter). In other particularly preferred embodiments, the at least one promoter is an Arabidopsis thaliana promoter or a cauliflower mosaic virus (CaMV) promoter, in particular the CaMV35S promoter. In another preferred variant, the at least one promoter is a sucrose synthase promoter.

The overexpression, which is preferred in accordance with the invention, of the vacuolar pyrophosphatase, preferably under the control of at least one CaMV35S promoter, leads to a markedly improved energizing of the vacuole, that is to an increase in the pH gradient, with this principally leading to an increase in the accumulation of storage substances, in particular of sucrose, in the vacuole; this is principally because the active transport of sucrose into the lumen of the vacuole is increased by the acidification of the vacuole, which is in turn occasioned by the overexpression which is preferred in accordance with the invention.

In addition to this, the overexpression, which is preferred in accordance with the invention, of the C-PPase results, in particular, in the breakdown of cytosolic or nuclear pyrophosphatate (PPᵢ) being increased substantially as compared with an untransformed beet cell. The substantial reduction, which has been brought about in this way, in the quantity of cytosolic or nuclear pyrophosphate results in PPᵢ-dependent sucrose breakdown being reduced or in meristem activity being increased as a result of the activation of different synthetic activities (see above). Together with the accumulation, which is increased by the overexpression of the V-PPase, of storage substances, in particular sucrose, in the vacuole, the sucrose content of the transgenic beet which can be obtained in accordance with the invention is preferably already increased prior to harvesting, that is while the plant is growing.

The present invention also relates to a nucleic acid molecule which encodes, preferably in accordance with the universal genetic standard code which is known per se, a protein having the biological activity of a soluble pyrophosphatase, in particular from Beta vulgaris, in particular a cytosolic pyrophosphatase (C-PPase), with the sequence of this nucleic acid molecule being selected from the group consisting of
a) a nucleotide sequence depicted in sequence ID No. 1, the sequence which is complementary thereto,
b) a nucleotide sequence which encodes the amino acid sequence depicted in sequence ID No. 2, and also its complementary nucleotide sequence, and
c) a modified nucleotide sequence, with a modified nucleic acid molecule of the modified nucleotide sequence hybridizing with the nucleic acid molecule having the nucleotide sequence in accordance with a) or b) and, in this connection, exhibiting a sequence identity of more than 80%, preferably more than 90%, 95% or 99%.

The present invention furthermore relates to a nucleic acid molecule which encodes, preferably in accordance with the universal genetic standard code which is known per se, a protein having the biological activity of a vacuolar pyrophosphatase, in particular from Beta vulgaris, with the sequence of this nucleic acid molecule being selected from the group consisting of
a) a nucleotide sequence depicted in sequence ID No. 4, the sequence which is complementary thereto,
b) a nucleotide sequence which encodes the amino acid sequence depicted in sequence ID No. 5, and also its complementary nucleotide sequence, and
c) a modified nucleotide sequence, with a modified nucleic acid molecule of the modified nucleotide sequence hybridizing with the nucleic acid molecule having the nucleotide sequence in accordance with a) or b) and, in this connection, exhibiting a sequence identity of more than 80%, preferably more than 90%, 95% or 99%.

The present invention furthermore relates to a nucleic acid molecule which encodes, preferably in accordance with the universal genetic standard code which is known per se, a promoter of vacuolar pyrophosphatase (V-PPase), in particular from Beta vulgaris, with the sequence of the nucleic acid molecule being selected from the group consisting of
a) a nucleotide sequence depicted in sequence ID No. 6, the sequence which is complementary thereto,
b) a nucleotide sequence depicted in sequence ID No. 7, the sequence which is complementary thereto, and
c) a modified nucleotide sequence, with a modified nucleic acid molecule of the modified nucleotide sequence hybridizing with the nucleic acid molecule according to a) or b) and, in this connection, exhibiting a sequence identity of more than 80%, 90%, 95% or 99%.

In this connection, the nucleic acid molecule is preferably a DNA molecule, for example cDNA or genomic DNA, or an RNA molecule, for example mRNA. The nucleic acid molecule is preferably derived from the sugar beet Beta vulgaris. The nucleic acid molecule is preferably present in isolated and purified form.

The invention consequently also encompasses modified nucleic acid molecules having a modified nucleotide sequence, which nucleic acid molecules can be obtained, for example, by the substitution, addition, inversion and/or deletion of one or a few bases in a nucleic acid molecule according to the invention, in particular within the coding sequence of a nucleic acid, that is nucleic acid molecules which can be described as being mutants, derivatives or functional equivalents of a nucleic acid molecule according to the invention. These manipulations of the sequences are, for example, carried out in order to selectively alter the amino acid sequence which is encoded by a nucleic acid. For example, the modified nucleic acids which are preferred in accordance with the invention encode altered enzymes, in particular altered vacuolar and/or cytosolic pyrophosphatases, and/or, in particular, with altered enzymic activity, and are used, in particular, for transforming plants which are used agriculturally, for the principal purpose of producing transgenic plants. According to the invention, these modifications preferably also have the aim of providing suitable restriction cleavage sites within the nucleic acid sequence or of removing nucleic acid sequences or restriction cleavage sites which are not required. In this connection, the nucleic acid molecules according to the invention are inserted into plasmids and subjected to a mutagenesis, or a sequence alteration by recombination, using standard methods of microbiology or molecular biology in a manner known per se.

Methods for in-vitro mutagenesis, “primer repair” method and restriction and/or ligation methods are, for example, suitable for generating insertions, deletions or substitutions, such as transitions and transversions (cf. Sambrook et al., Molecular Cloning: A Laboratory Manual, 2nd edition (1989), Cold Spring Harbor Laboratory Press, NY, USA). Sequence alterations can also be achieved by attaching natural or synthetic nucleic acid sequences. Examples of synthetic nucleic acid sequences are adaptors or linkers which, inter alia, are also added onto nucleic acid fragments in order to link these fragments together. The present invention also relates to naturally occurring sequence variants of the nucleic acid molecules according to the invention or the nucleic acid molecules which are used in accordance with the invention.

The phrases analogous to the phrase “modified nucleic acid molecule which hybridizes with a nucleic acid molecule” which are used in connection with the present invention mean that a nucleic acid molecule hybridizes, under moderately stringent conditions in a manner known per se, with another nucleic acid molecule which is different therefrom. For example, the hybridization can take place with a radioactive gene probe in a hybridization solution (for example: 25% formamide, 5×SSPE, 0.1% SDS, 5× Denhardt's solution, 50 mg of herring sperm DNA/ml, as regards the composition of the individual components) at 37° C. for 20 hours (cf. Sambrook et al., Molecular Cloning: A Laboratory Manual, 2nd edition (1989), Cold Spring Harbor Laboratory Press, NY, USA). The probe which is bound nonspecifically is then removed, for example, by washing the filters several times in 2×SSC/0.1% SDS at 42° C. Preference is given to washing with 0.5×SSC/0.1% SDS, particularly preferably with 0.1×SSC/0.1% SDS, at 42° C. These hybridizing nucleic acid molecules, which are preferred in accordance with the invention, exhibit, in a preferred embodiment, at least 80%, preferably at least 85%, 90%, 95%, 98% and, particularly preferably, at least 99%, homology, that is sequence identity at the nucleic acid level, with each other.

In this connection, the expression “homology” describes the degree of relatedness between two or more nucleic acid molecules, with this degree being determined by the congruence between their nucleotide sequences. The “homology” percentage ensues from the percentage of congruent regions in two or more sequences, taking into consideration gaps or other sequence peculiarities. The nucleic acid molecule nucleotide sequences which are to be compared are preferably compared, for this purpose, over the whole of their length.

Methods, which are preferred and known per se, for determining homology, which methods are principally realized in computer programs, initially generate the greatest degree of congruence between the sequences being compared, with examples of these methods being the GCG program package, including GAP (Devereux, J., et al., Nucleic Acids Research, 12 (12) (1984), 387; Genetics Computer Group University of Wisconsin, Madison (WI)); BLASTP, BLASTN and FASTA (Altschul, S., et al., J. Molec Bio 215 (1990), 403-410). The known Smith Waterman algorithm can also be used for determining the homology. The choice of the program depends both on the comparison to be carried out and on whether the comparison is being carried out between sequence pairs, when GAP or Best Fit is preferred, or between a sequence and an extensive sequence database, when FASTA or BLAST is preferred.

The present invention also relates to a vector which is preferably employed in the process according to the invention and which contains at least one of the sequences of the above-mentioned nucleic acid molecules according to the invention. Preference is given, according to the invention, to this vector being a viral vector. In another variant, this vector is preferably a plasmid and, in a particularly preferred version, the vector pBinAR. One variant preferably also encompasses the vectors in which the at least one nucleic acid molecule according to the invention which they contain is operatively linked to at least one regulatory element which ensures that translatable nucleic acid molecules are transcribed and synthesized in prokaryotic and/or eukaryotic cells. Regulatory elements of this nature are preferably promoters, enhancers, operators and/or transcription termination signals. The above-mentioned vectors according to the invention preferably also contain antibiotic resistance genes, herbicide resistance genes and/or other customary selection markers.

The present invention furthermore relates to a host cell which has been transformed with at least one of the above-mentioned vectors according to the invention, with this host cell preferably being a bacterial cell, a plant cell or an animal cell. The present invention therefore also relates to a transgenic and, preferably, fertile plant which is obtained using the process according to the invention, with at least one of the cells of this plant being transformed and this plant preferably being characterized by an increased content of sucrose and/or an increased growth as a consequence of increased meristem activity. The invention naturally also encompasses the progeny and further strains which are obtained from the transformed plants according to the invention.

The present invention also relates to transgenic plant cells which have been transformed, that is transfected, with one or more nucleic acid molecule(s) according to the invention or nucleic acid molecule(s) which is/are used in accordance with the invention, and also to transgenic plant cells which are derived from transformed cells of this nature. These cells contain one or more nucleic acid molecule(s) which is/are used in accordance with the invention or nucleic acid molecule(s) according to the invention, with this/these molecules preferably being linked to regulatory DNA elements which ensure transcription in plant cells. These cells can be distinguished from naturally occurring plant cells by the fact, in particular, that they contain at least one nucleic acid molecule according to the invention or nucleic acid molecule which is used in accordance with the invention which does not occur naturally in these cells and/or by the fact that such a molecule is integrated at a site in the genome of the cell at which it does not naturally occur, that is in a different genomic environment, or is present in a copy number which is different from the natural copy number and/or is under the control of at least one different promoter.

The transgenic plant cells can be regenerated into whole plants using techniques which are known to the skilled person. The plants which can be obtained by regenerating the transgenic plant cells according to the invention likewise form part of the subject matter of the present invention. The invention also relates to plants which contain at least one cell, preferably, however, a multiplicity of cells, which contain(s) the vector systems according to the invention, or the vector systems which are used in accordance with the invention, and also derivatives or parts thereof, and which, as a result of having taken up these vector systems, derivatives or parts thereof, are capable of synthesizing polypeptides (proteins) which bring about a modification of pyrophosphatase activity. The invention consequently makes it possible to provide plants of a very wide variety of species, genera, families, orders and classes which exhibit the above-mentioned characteristics, in particular. The transgenic plants according to the invention are in principle monocotyledonous or dicotyledonous plants such as Graminae, Pinidae, Magnoliidae, Ranunculidae, Caryophyllidae, Rosidae, Asteridae, Aridae, Liliidae, Arecidae and Commelimidae, and also Gymnospermae, as well as algae, mosses and ferns, or else calli, plant cell cultures, etc., and also parts, organs, tissues and harvesting or propagation materials thereof. However, the plants are preferably productive plants, in particular sucrose-synthesizing and/or storing plants such as sugar beet.

The present invention also relates to harvesting material and propagation material from the above-mentioned transgenic plants according to the invention, for example flowers, fruits, seeds, tubers, roots, leaves, rhizomes, seedlings, cuttings, etc.

The present invention also relates to the use of at least one of the above-mentioned nucleic acid molecules according to the invention for producing such an above-mentioned transgenic plant which contains at least one transformed cell, in particular in combination with at least one of the above-mentioned vectors.

The sequence listing is part of this description and explains the present invention; it contains the sequences having the SEQ ID Nos. 1 to 7:
SEQ ID No. 1 shows the DNA sequence, comprising 1041 nucleotides, of the Beta vulgaris nucleic acid molecule (bsp1) encoding the soluble beta-pyrophosphatase;
SEQ ID No. 2 shows the polypeptide sequence, comprising 222 amino acids, of the Beta vulgaris soluble beta-pyrophosphatase (BSP1);
SEQ ID No. 3 shows the polypeptide sequence, comprising 245 amino acids, of a recombinant soluble beta-pyrophosphatase in vector pQE30 having an N-terminal His tag;
SEQ ID No. 4 shows the DNA sequence, comprising 2810 nucleotides, of the isoform I Beta vulgaris nucleic acid molecule (bvp1) encoding the vacuolar beta-pyrophosphatase;
SEQ ID No. 5 shows the polypeptide sequence, comprising 764 amino acids, of the isoform I Beta vulgaris vacuolar beta-pyrophosphatase (BVP1);
SEQ ID No. 6 shows the DNA sequence, comprising 1733 nucleotides, of the bvp1 promoter for the isoform I Beta vulgaris vacuolar beta-pyrophosphatase;
SEQ ID No. 7 shows the DNA sequence, comprising 962 nucleotides, of the bvp2 promoter for the isoform II Beta vulgaris vacuolar beta-pyrophosphatase.
SEQ ID No. 8 shows the DNA sequence, comprising 18 nucleotides, of the sense primer in accordance with example 1.
SEQ ID No. 9 shows the DNA sequence, comprising 22 nucleotides, of the antisense primer in accordance with example 1.
SEQ ID No. 10 shows the DNA sequence, comprising 38 nucleotides, of the sense primer in accordance with example 2.
SEQ ID No. 11 shows the DNA sequence, comprising 38 nucleotides, of the antisense primer in accordance with example 2.
SEQ ID No. 12 shows the DNA sequence, comprising 31 nucleotides, of the sense primer in accordance with example 4.
SEQ ID No. 13 shows the DNA sequence, comprising 31 nucleotides, of the antisense primer in accordance with example 4.
SEQ ID No. 14 shows the DNA sequence, comprising 30 nucleotides, of the sense primer in accordance with example 5.
SEQ ID No. 15 shows the DNA sequence, comprising 31 nucleotides, of the antisense primer in accordance with example 5.
SEQ ID No. 16 shows the DNA sequence, comprising 34 nucleotides, of the sense primer in accordance with example 6.
SEQ ID No. 17 shows the DNA sequence, comprising 35 nucleotides, of the antisense primer in accordance with example 6.
SEQ ID No. 18 shows the DNA sequence, comprising 20 nucleotides, of a sense primer in accordance with example 7.
SEQ ID No. 19 shows the DNA sequence, comprising 21 nucleotides, of an antisense primer in accordance with example 7.
SEQ ID No. 20 shows the DNA sequence, comprising 24 nucleotides, of a sense primer in accordance with example 7.
SEQ ID No. 21 shows the DNA sequence, comprising 20 nucleotides, of an antisense primer in accordance with example 7.

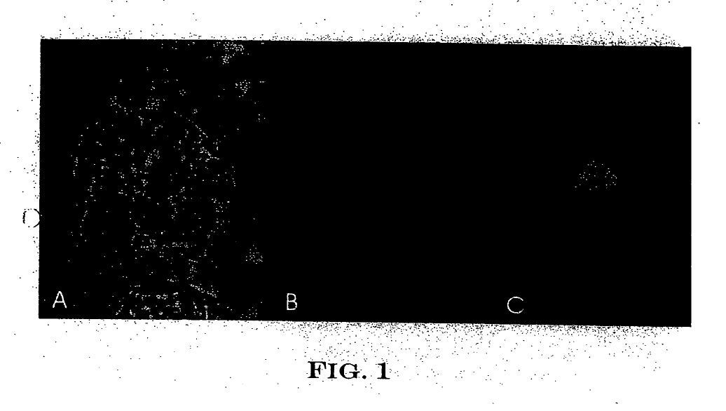

The present invention is explained in more detail by the FIGS. 1 to 10 and the following examples.

FIG. 1 shows fluorescence-microscopic photographs of transformed beet cells: FIG. 1a shows a transformed Beta vulgaris cell in transmitted light, FIG. 1b shows the subcellular location of the RFP control plasmid in the plastids and FIG. 1c shows the subcellular location of GFP-fused soluble pyrophosphatase (BSP1) in the cytoplasmic and nucleus-proximal regions of the protoplasts;

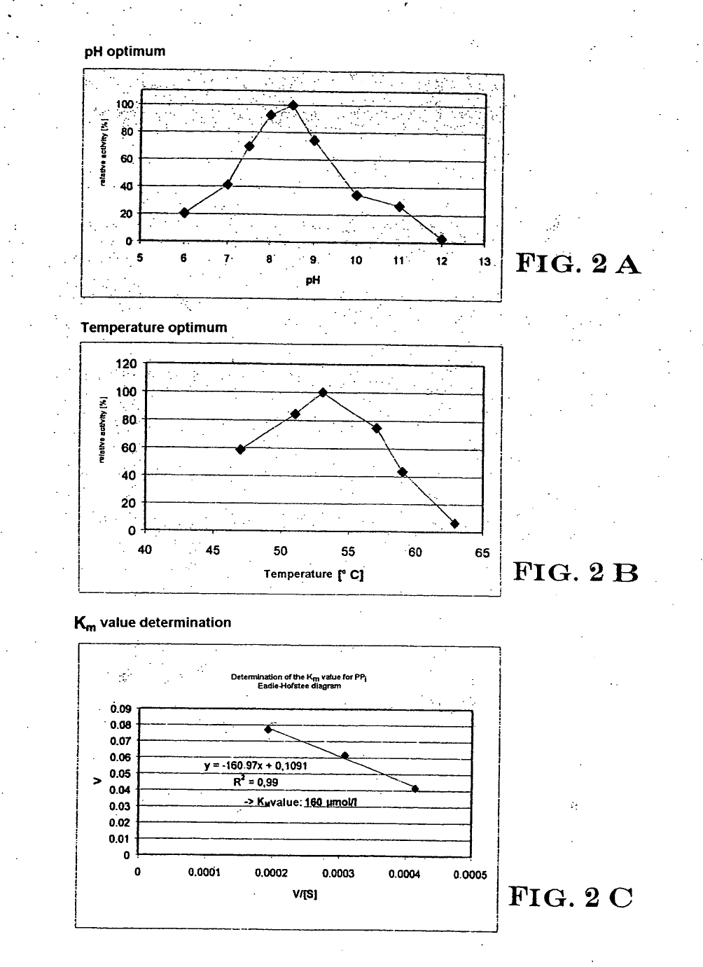

FIG. 2 shows biochemical properties of the soluble beta-pyrophosphatase (BSP1): FIG. 2a shows the pH dependence, and FIG. 2b shows the temperature dependence, of the enzyme activity while FIG. 2c shows the determination of the Kₘ value for pyrophosphate (Eadie-Hofstee diagram);

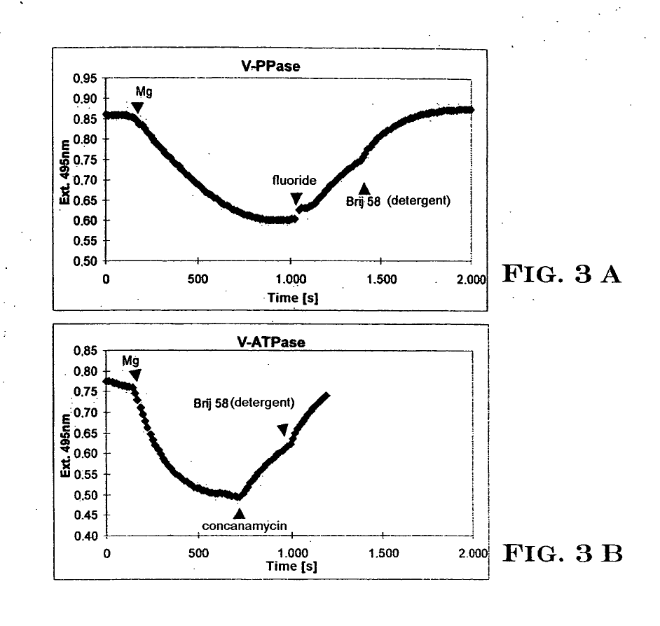

FIG. 3 shows the proton pump activity in beets which have been stored for three months: FIG. 3a shows the V-PPase activity while FIG. 3b shows the V-ATPase activity;

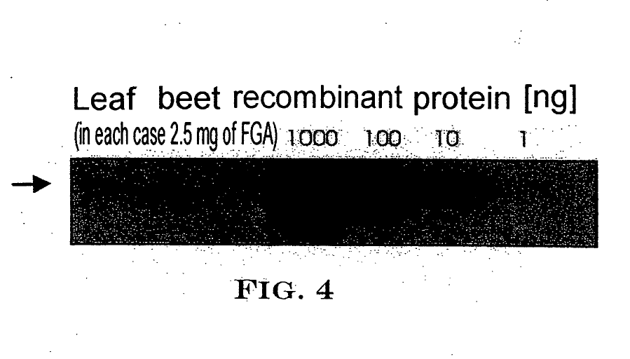

FIG. 4 shows a Western blot analysis of BSP1 in leaf and beet;

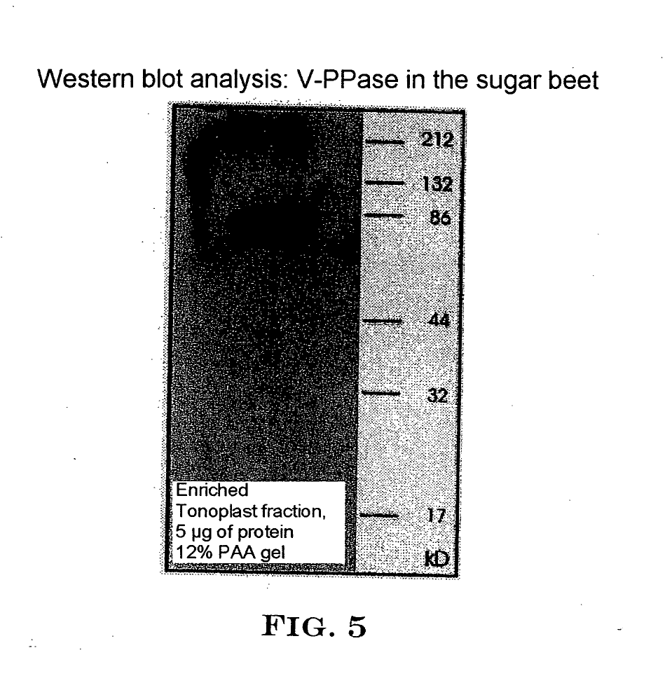

FIG. 5 shows a Western blot analysis of V-PPase in sugar beet (Beta vulgaris);

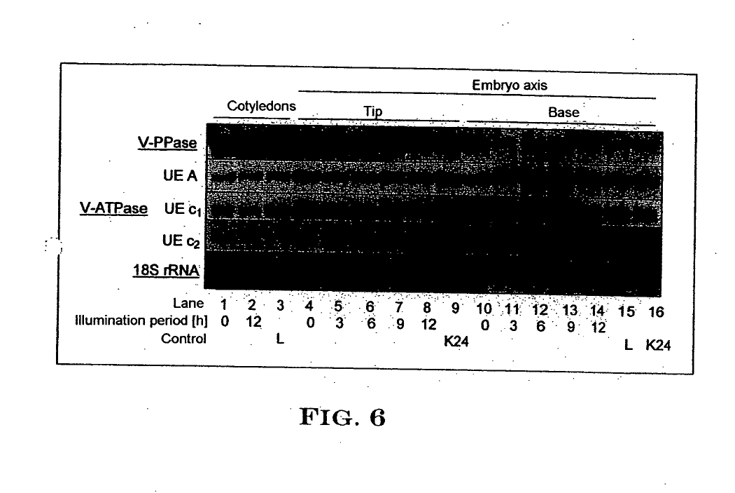

FIG. 6 shows the Northern blot analysis of V-PPase and V-ATPase in sugar beet seedlings;

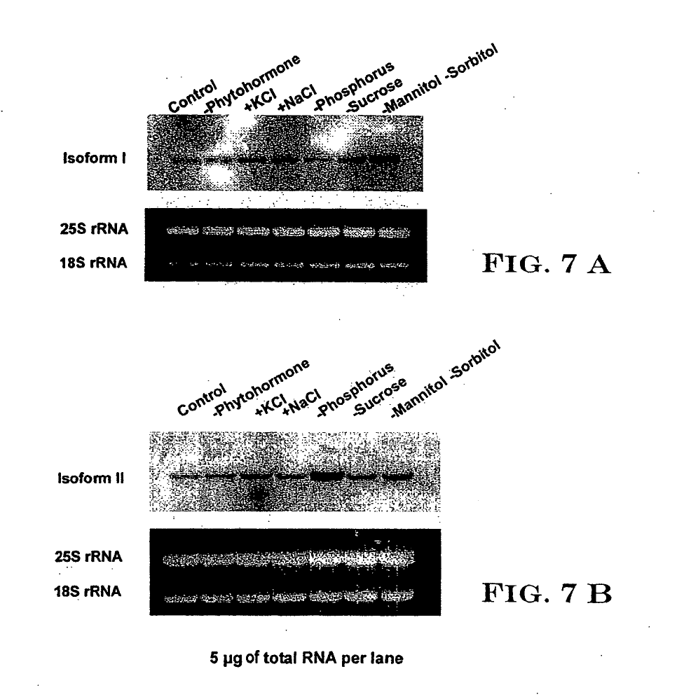

FIG. 7 shows the Northern blot analysis of V-PPase when sugar beet cells in suspension culture are stress-treated;

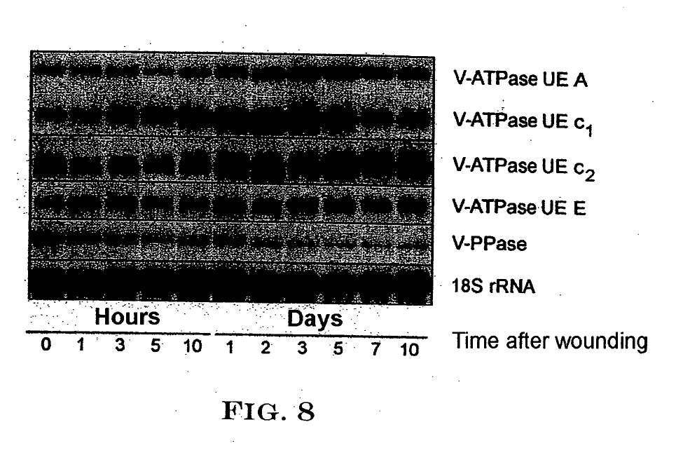

FIG. 8 shows the Northern blot analysis of the expression pattern after wounding sugar beets;

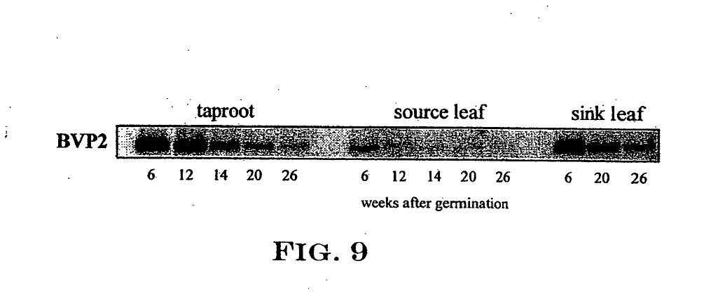

FIG. 9 shows the Northern blot analysis of the development-dependent expression of the isoform II Beta vulgaris V-PPase polypeptide (BVP2);

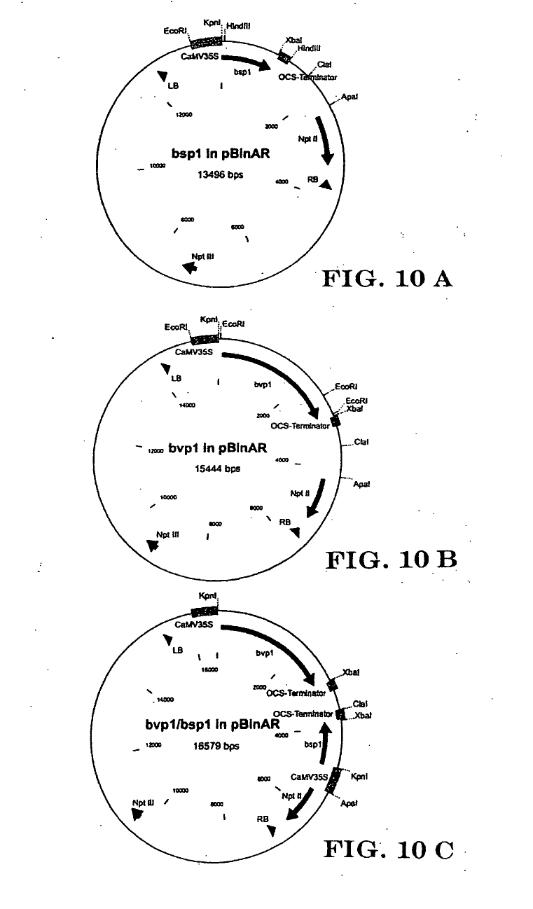

FIG. 10 shows diagrams of the structures of recombinant vectors: FIG. 10a shows the vector which is obtained in accordance with example 4, FIG. 10b shows the vector which is obtained in accordance with example 5 and

FIG. 10c shows the vector which is obtained in accordance with example 6.

### EXAMPLE 1

### Isolating the cDNA sequence for a soluble pyrophosphatase from Beta vulgaris L. (BSP1)

The total RNA was isolated from Beta vulgaris L. cells in suspension culture in accordance with Logemann et al. (Analyt. Biochem., 163 (1987), 16-20) and transcribed into cDNA using reverse transcriptase. Degenerate primers were prepared on the basis of sequence comparisons and then used to amplify, by means of PCR, a 435 bp partial cDNA sequence from the region encoding the sugar beet soluble pyrophosphatase (bsp1):

Sense primer:

(SEQ ID No. 8)

TGC TGC TCA TCC WTG GCA
Antisense primer:

(SEQ ID No. 9)

TCR TTY TTC TTG TAR TCY TCA A

RLM-RACE technology (GeneRacer™ kit, Invitrogen, Groningen, Netherlands) was then used to determine the sequence of the bsp1 full-length cDNA (1041 bp) (SEQ ID No. 1), which, according to this determination, consists of a 666 bp ORF which is flanked by a 118 bp 5′-UTR and a 257 bp 3′-UTR. The amino acid sequence encoded by the bsp1 cDNA ORF is depicted in SEQ ID No. 2 and possesses 222 amino acids.

Tables 1 and 2 show biochemical properties of BSP1 and the effect of doubly charged cations on the activity of the BSP1:

TABLE 1
Biochemical properties of BSP1

| | |
|---|---|
| Amino acids | 222 |
| Size | 25.5 kDa |
| pI (calculated) | 5.62 |
| Degree of oligomerization* | possible tetramer (gel |
| | filtration) |
| pH optimum* | pH 8.5 |
| Temperature optimum* | 53° C. |
| Kₘ PPᵢ (2.5 mmol of Mg/l)* | ˜160 μmol/l |
| Doubly charged cations* | Mg²⁺ essential, |
| | Ca²⁺ (competitively) inhibiting |

*determined using the recombinant protein, pQE30 vector (Qiagen ®, Hilden, Germany) having an N-terminal HIS tag; the amino acid sequence is depicted in SEQ ID No. 3. The same primers as those described in example 2 (SEQ ID Nos. 10 and 11) were used for amplifying the coding region of bsp1.

TABLE 2
Effect of doubly charged ions on the activity of BSP1

| Magnesium | Calcium | Relative |
|---|---|---|
| conc. | conc. | pyrophosphatase |
| [mmol/l] | [mmol/l] | activity [%] |
| 2.5 | 0 | 100 |
| 2.5 | 0.05 | 55 |
| 2.5 | 0.5 | 6 |
| 0 | 0 | 0 |

Results:

FIG. 2a shows the results of the pH determination (pH 8.5), while FIG. 2b shows the results of the temperature optimum determination (53° C.) and FIG. 2c shows the results of the K_{M} value determination (160 μmol of PPᵢ/l)

### EXAMPLE 2

### Investigations into the Subcellular Location of BSP1

In addition to the computer-assisted analysis of the primary BSP1 sequence with regard to signal peptides, the coding region was cloned into a modified pFF₁₉G vector (Timmermanns et al., J. Biotech. 14 (1990), 333-344) which, instead of the β-glucoronidase structural gene, carries the sequence for the green fluorescent protein (GFP) (Sheen, et al., Plant J. 8(5) (1995), 777-784). The sense primer which is used for this contains, in addition to a BamHI cleavage site (underlined), a “Kozak” sequence immediately upstream of the start ATG in order to ensure optimal translation. The antisense primer contains both a PstI cleavage site and an SalI cleavage site (underlined):

Sense primer:

(SEQ ID No. 10)

GTC GGG ATC CGC CAC CAT GGA TGA GGA GAT GAA TGC TG
Antisense primer:

(SEQ ID No. 11)

GAA GCT GCA GGT CGA CTC TCC TCA ATG TCT GTA GGA TG

The ligation was carried out after both the bap1 amplificate and the pFF₁₉G vector had been cut with BamHI and PstI, after which Beta vulgaris cells in suspension culture were biolistically transformed using a particle cannon (Biolistic® PDS-1000/He, BioRad, Hercules, Calif., USA). In this connection, a pFF₁₉G control plasmid which contained the sequence for a fusion protein composed of an 81-amino acid peptide from the Brassica juncea plastid γ-ECS and the Discosoma spec. red fluorescent protein (dsRED) (Jach et al., Plant J. 28(4) (2001), 483-491) was introduced at the same time. 24 h after the bombardment, the cell walls were digested using lytic enzymes and, after a further 24 h, the transient expression of the two fusion proteins in the protoplasts was investigated by fluorescence microscopy using an inverse light microscope. The GFP fusion protein was analyzed using an FITC filter (excitation: 450-490 nm, emission: 515 nm long pass), while, in the case of the dsRED fusion protein, an XF137-2 filter (excitation: 540±30 nm, emission: 585 nm long pass) was used.

Results:

FIG. 1 shows the subcellular location of BSP1 as determined by the fluorescence-microscopic GFP analysis of transformed beet cells: it can be seen from FIG. 1a that a transformed Beta vulgaris cell cannot be distinguished from an untransformed Beta vulgaris cell. FIG. 1b relates to the RFP control plasmid. It can be seen that the plastids light up (bright) red due to the plastid signal peptide of the plastid γ-ECS. In FIG. 1c, the excitation of the GFP shows that the soluble pyrophosphatase which is fused with the GFP does not possess any plastid signal peptide. The cytoplasmic and nuclear localization in the protoplast can be clearly seen. BSP1 is evidently a soluble pyrophosphatase which is located in the cytosol or the nucleus. This pyrophosphatase is also termed C-PPase.

### EXAMPLE 3

### Detecting Function by Overexpressing BSP1 in E. coli

The sequence encoding Beta vulgaris C-PPase (BSP1) was amplified by means of PCR. The primers which were used for this purpose were the same as used for the above-described amplification for the pFF₁₉::GFP construct (example 2).

Cloning into the expression vector pQE30 (Qiagen®, Hilden) took place by way of BamHI/SalI. The construct was transformed into E. coli-DH5α cells together with a pUBS520-plasmid (Brinkmann et al., Gene 85(1) (1989), 109-114).

The production of BSP1 was induced with 1 mmol of IPTG (isopropyl-β-thiogalactopyranoside)/l after the bacteria had reached a density of OD₆₀₀=1. Growth took place overnight at 37° C. The BSP1 was purified under native conditions. The cells were disrupted using a French press. The lysis buffer which was used in this connection contained 50 mmol of MOPS (pH 8)/l, 300 mmol of NaCl/l, 10 mmol of imidazole/l and 5 mmol of MgCl₂/l. Following the binding, mediated by the 6 N-terminal histidines, to a nickel-NTA matrix, several washing steps were carried out using an increasing concentration of imidazole (20-75 mmol/l) under what were otherwise identical buffer conditions. Elution was effected analogously using 100-250 mmol of imidazole/l.

For the activity assay, 200 μl of reaction buffer (standard: 50 mmol of Tris (pH 8.5)/l, 1 mmol of pyrophosphate/l, 2.5 mmol of MgCl₂/l) were added to 50 μl of protein solution and the whole was incubated for 15 min. The reaction was stopped with 750 μl of dye solution (3.4 mmol of ammonium molybdate/l in 0.5 mol of sulfuric acid/l, 0.5 mol of SDS/l, 0.6 mol of ascorbic acid/l: 6:2:1, v/v/v). After 20 min, the absorption was measured at 820 nm (Rojas-Beltrán et al. 39 (1999), 449-461).

### EXAMPLE 4

### Cloning the Soluble BSP1 Pyrophosphatase (C-PPase) into the Transformation Vector pBinAR

Using the primers which are specified below and the cDNA, which is described above, from cells in suspension culture, the 1041 bp full-length cDNA (SEQ ID No. 1) for the soluble pyrophosphatase (BSP1) was amplified by means of PCR. The ends of the primers were provided with KpnI (sense primer) and, respectively, XbaI (antisense primer) cleavage sites (underlined) in order to be subsequently able to ligate the amplificate into the above-described plant transformation vector pBinAR (Höfgen and Willmitzer, Plant Science 66 (2) (1990), 221-230).

Sense primer:

(SEQ ID No. 12)

CCG GGG TAC CAA GGA ATT TGT AGA TCT CCG A
Antisense primer:

(SEQ ID No. 13)

CTA GTC TAG AAG CCT CCT AAA CCA AAC ATG A

The resulting vector is depicted in FIG. 10a.

### EXAMPLE 5

### Cloning the Vacuolar Pyrophosphatase (V-PPase) into the Transformation Vector pBinAR

The following primers, which bind at the beginning of the 5′-UTR (sense primer) and at the end of the 3′-UTR (antisense primer) of the isoform I of the sugar beet V-PPase were generated (Kim et al., Plant. Physiol. 106 (1994), 375-382):

Sense primer:

(SEQ ID No. 14)

ACA CTC TTC CTC TCC CTC TCT TCC AAA CCC
Antisense primer:

(SEQ ID No. 15)

TAG ATC CAA TCT GCA AAA TGA GAT AAA TTC C

Using these primers, the V-PPase sequence (bvp1) was amplified from the above-described total cDNA by means of PCR and the 2860 bp amplificate (SEQ ID No. 4) was then cloned, as an intermediate cloning, into the vector pCR®2.1-TOPO® (Invitrogen, Groningen, Netherlands). The resulting amplificate contains the beta-V-PPase (BVP1)-encoding region (SEQ ID No. 5).

The KpnI and XbaI restriction cleavage sites which were located to the left and right of the insertion site in the TOPO vector were used to excise the sequence of the V-PPase and then ligate it into the MCS of the plant transformation vector pBinAR, which was likewise cut with KpnI and XbaI. The vector which was obtained in this way is depicted in FIG. 10b.

### EXAMPLE 6

### Preparing the Double Construct by Cloning the Sequences for V-PPase and C-PPase into pBinAR

The entire C-PPase expression cassette was amplified from the corresponding pBinAR construct by means of PCR. In addition to the full-length cDNA for C-PPase, it contains the CaMV35S promoter (540 bp) and the OCS terminator (196 bp). The sense primer which was used for the amplification binds to the 5′ end of the CaMV35S promoter and possesses an ApaI cleavage site, while the antisense primer attaches to the 3′ end of the OCS terminator and possesses a ClaI cleavage site (underlined):

Sense primer:

(SEQ ID No. 16)

AAG TCG GGG CCC GAA TTC CCA TGG AGT CAA AGA T
Antisense primer:

(SEQ ID No. 17)

GAA GCC ATC GAT AAG CTT GGA CAA TCA GTA AAT TG

The amplificate which was obtained using these primers was digested with ApaI and ClaI and then ligated into the V-PPase/pBinAR construct which was likewise digested with ApaI and ClaI. In the construct, these two cleavage sites are located between the OCS terminator and the right-hand border region of the T-DNA. Due to the positions of the ApaI and ClaI cleavage sites, the two expression cassettes are consequently in opposite orientations in the pBinAR double construct. The double vector is depicted in FIG. 10c.

### EXAMPLE 7

### Cloning the V-PPase Promoters

The promoter sequence (SEQ ID No. 6) of the isoform I V-PPase (BSP1) was isolated using a genomic DNA library which had been prepared with the aid of the Lambda-ZAP-XhoI-Partial-Fill-In® vector kit (Stratagene, Amsterdam, Netherlands) (Lehr et al., Plant Mol. Biol., 39 (1999), 463-475). A 569 bp sequence from the coding region, which sequence had been prepared using degenerate primers:

Sense primer:

(SEQ ID No. 18)

GGW GGH ATT GCT GAR ATG GC
Antisense primer:

(SEQ ID No. 19)

AGT AYT TCT TDG CRT TVT CCC

was used as the biotin probe.

The promoter sequence (SEQ ID No. 7) of the isoform II (BSP2) was determined by means of inverse PCR. Genomic DNA was isolated from sugar beet leaves using the method of Murray and Thompson (Nucl. Acids Res. 8 (1980), 4321-4325). Following digestion with the restriction enzyme TaqI, the ends of the cleavage products were ligated so as to form circular DNA molecules. These were used as templates in a PCR, with the sense primer originating from the 5′-proximal region of the coding region and the antisense primer originating from the 5′-UTR:

Sense primer:

(SEQ ID No. 20)

CCA AAA CGT CGT CGC TAA ATG TGC
Antisense primer:

(SEQ ID No. 21)

ACC GGA ACC CTA ACT TTA CG

### EXAMPLE 8

### Activity of the V-PPase

a) Tonoplast Isolation

Tonoplasts were isolated from sugar beets following the method of Ratajczak et al. (Planta, 195 (1995), 226-236). 45 g of beet material (stored for 4 months at 4° C.) were comminuted in 160 ml of homogenization medium (pH 8.0), 450 mmol of mannitol/l, 200 mmol of tricine/l, 3 mmol of MgSO₄/l, 3 mmol of EGTA/l, 0.5% (w/v) polyvinylpyrrolidone (PVP), 1 mmol of DTT/l) in a mixer. The homogenate was filtered through 200 μm gauze and then centrifuged at 4200×g for 5 min. The supernatant was centrifuged at 300 000×g for 30 min in a Beckman® 50.2 Ti rotor in order to obtain the microsomal fraction. The resulting pellets were resuspended in 50 ml of homogenization medium. In each case 25 ml were underlaid with 8 ml of gradient medium (5 mmol of HEPES (pH 7.5)/l, 2 mmol of DTT/l and 25% (w/w) sucrose) and centrifuged at 100 000×g for 90 min. In each case 1 ml of interphase, which represents the tonoplast fraction, was removed from both gradients using a Pasteur pipette and mixed with dilution medium (50 mmol of HEPES (pH 7.0)/l, 3 mmol of MgSO₄/l and 1 mmol of DTT/l). The tonoplasts were then pelleted at 300 000×g for 30 min, resuspended in 500 μl of storage medium (10 mmol of HEPES (pH 7.0)/l, 40% glycerol, 3 mmol of MgSO₄/l and 1 mmol of DTT/l) and frozen in liquid nitrogen. The subsequent storage was at −80° C.

B) Detecting the Proton Pump Activity

The V-PPase proton pump activity was determined in accordance with Palmgren (Plant Physiol., 94 (1990), 882-886). 50 μg of tonoplast protein were used.

Results:

FIGS. 3a and 3b show the H⁺ pump activity in beets which had been stored for three months:
The specific activity of the V-ATPase is about twice as high as that of the V-PPase.
The vesicular acidification leads to comparable pH gradients.

### EXAMPLE 9

### Antisera and Immunoblot Analysis

A rabbit polyclonal antiserum directed against the mung bean (Vigna radiata) V-PPase was used to detect the Beta vulgaris V-PPase proteins (Maeshima and Yoshida, J. Biol. Chem., 264 (1989), 20068-20073). A rabbit antibody directed against the holoenzyme of the Kalanchoe diagremontiana vacuolar adenosine triphosphatase (V-ATPase) was used to detect the V-ATPase proteins (Haschke et al., In: Plant Membrane Transport, Editors: Dainty, J., De Michelis, M. I., Marré, E. and Rasi-Caldogno, F., 1989, 149-154, Elsevier Science Publishers B. V., Amsterdam).

In the case of the C-PPase, use was made of a rabbit polyclonal antiserum which had been prepared by the company Eurogentec (Herstal, Belgium). In this connection, the recombinant BSP1 protein which had been purified by means of Ni-NTA affinity chromatography was injected as the antigen.

Immunoblot analyses were carried out as described in Weil and Rausch (Planta, 193 (1994), 430-437). Differently from this method, 5% skimmed milk powder was used instead of 8% BSA for the blocking. “SuperSignal West Dura®” (Pierce, Rockford, USA) was used as substrate.

In order to detect V-PPase and V-ATPase, in each case 5 μg of protein from the enriched tonoplast fraction were fractionated electrophoretically in a native 12% polyacrylamide gel. In the case of the C-PPase, in each case 0.5 g of leaf and beet material were triturated in liquid nitrogen and the homogenate was taken up directly in 1 ml of reducing 2× loading buffer (RotinLoad1, Roth, Karlsruhe). In each case 5 μl of crude extract (corresponds to 2.5 mg of fresh weight equivalent) were fractionated in a 15% polyacrylamide gel.

Results:

FIG. 4 shows the results of a Western blot analysis for BSP1:

BSP1 is present both in the beet and in the leaf.

FIG. 5 shows the results of a Western blot analysis in the case of V-PPase:

The V-PPase can be detected in the Beta vulgaris beet.

### EXAMPLE 10

### RNA Extraction and Northern Blot Analysis

Beta vulgaris cells in suspension culture were grown in “Gamborg B₅” medium containing 2% sucrose in the added presence of the following phytohormones: 0.2 mg of kinetin/l, 0.5 mg of naphthyl acetic acid (NAA)/l, 0.5 mg of indole-3-acetic acid (IAA)/l and 2 mg of 2,4-dichlorophenoxyacetic acid (2,4-D)/l.

For the stress experiments, 6-day-old cells were firstly transferred to fresh medium and, after a further two days, transferred to 0.9% agar plates, where they were left for 3 days. While the plates contained Gamborg B₅ medium containing 2% sucrose, in the same way as the liquid medium, they additionally contained 125 mmol of mannitol/l and 125 mmol of sorbitol/l. Under stress conditions, the cells were grown on plates without mannitol and sorbitol, without phytohormones, without sucrose, without phosphate or with 100 mmol of KCl or NaCl/l.

For the investigations on seedlings, Beta vulgaris seeds (diploid hybrids, KWS, Einbeck) were sown in dishes containing moist sand. In order to protect against evaporation, the dishes were covered with a plastic hood and then stored in the dark at 23° C. (control plants germinated under light with a light/dark rhythm of 12/12 h). After 6 days, the plants which had germinated in the dark were exposed to the light and their embryo axis, which was subdivided into tip (upper 0.5 cm) and base, and also their cotyledons, were harvested at the times 0, 3, 6, 9 and 12 h after the beginning of the illumination. In order to be able to rule out development-dependent effects, some of the plants were left in the dark for a further 24 h before corresponding control samples were taken.

In order to investigate the development-dependent expression of the V-PPase, sugar beet were grown under outdoor conditions. Samples of different tissues were taken at intervals of several weeks and stored at −80° C. until worked up.

The sugar beet which were used for the wounding experiment were stored at 4° C. for 6 months after harvesting. The wounding was carried out as described by Rosenkranz et al. (J. Exp. Bot., 52 (2001), 2381-2384).

Total RNA was isolated using the method of Logemann et al. (Analyt. Biochem., 163 (1987), 16-20). In each case 15 μg of RNA were fractionated electrophoretically, per lane, in a 1.4% agarose gel having a formaldehyde content of 2%. The RNA was then transferred by capillary transfer to a Nylon membrane (Duralon, Stratagene, Amsterdam) and fixed on the membrane using UV light (Crosslinker®, Stratagene, Amsterdam). Detection was effected using biotin-labeled probes as described by Löw and Rausch (In: Biomethods; A laboratory guide to biotin-labelling in biomolecule analysis, Editors: Meier, T. and Fahrenholz, F., 1996, 201-213, Birkhäuser Verlag, Basle).

FIG. 6 shows a Northern blot analysis of V-PPase and V-ATPase transcripts in different tissues from 6-day-old, etiolated sugar beet seedlings which had been exposed, following their growth in the dark, to illumination periods of different lengths (0, 3, 6, 9 and 12 h, respectively). In order to control development-dependent changes, some dark germinators were left in the dark for a further 24 h, that is for a total of 7 days, in order to be able to compare their transcript quantities (lanes 9 and 15, respectively) with those of the 6-day-old etiolated seedlings without light contact (lanes 4 and 10, respectively). 6-day-old light germinators, which had grown under a 12/12 h light/darkness rhythm at 160 μmol photons per m²/s (lanes 3 and 16) served as a further control. In each case 15 μg of RNA were loaded.

Results:

FIG. 6 shows the results of a Northern blot analysis of the expression of V-PPase and V-ATPase in beta seedlings.
Irrespective of the degree of illumination, the V-PPase is strongly expressed in tissues exhibiting a high rate of division (embryo axis tip) or synthetic activity (cotyledons) whereas its expression is low in fully differentiated tissues (embryo axis base).
The subunits of the V-ATPase are expressed more weakly in the cotyledons than in the embryo axis base, with this being irrespective of the degree of illumination. While the expression is high in the actively dividing region of the embryo axis tips in the etiolated seedlings which have been grown in the dark, it decreases markedly only a few hours after illumination.

FIGS. 7a and 7b show the results of a Northern blot analysis of the effects of different stress treatments on the vacuolar pyrophosphatase (isoforms I and II) transcript levels in Beta vulgaris L cells in suspension culture.

FIG. 8 shows the results of a Northern blot analysis, from which it is evident that V-ATPase and V-PPase genes exhibit opposing expression patterns in beta beets following wounding.

Finally, FIG. 9 shows a Northern blot analysis of the development-dependent expression of vacuolar pyrophosphatase (isoform II=BVP2) in different Beta vulgaris tissues.

### EXAMPLE 11

### Expression of V-PPase and C-PPase in Arabidopsis thaliana

In order to investigate the effect of the overexpression of the Beta vulgaris cytosolic pyrophosphatase (C-PPase), the overexpression of Beta vulgaris vacuolar pyrophosphatase (V-PPase), or the simultaneous overexpression of both pyrophosphatases, on the growth, in particular the rosette growth, of Arabidopsis thaliana, transgenic Arabidopsis plants were in each case prepared using the above-mentioned processes according to the invention. The pBinAR vectors which were used for this purpose (FIGS. 10a-c) also contained the CaMV35S promoter in addition to the full-length cDNA for the respective pyrophosphatase. The respective pyrophosphatases were overexpressed under the control of this ³⁵S promoter. The effect on the rosette growth of Arabidopsis thaliana was examined in comparison with the wild type. This involved determining the dry weights of six-week-old plants (table 3).

TABLE 3

| | | | | V-PPase & |
|---|---|---|---|---|
| Arabidopsis | Wild | C-PPase | V-PPase | C-PPase |
| thaliana | type | sense | sense | sense |
| Total shoot dry weight | 100 ± 6 | 112 ± 8 | 118 ± 11 | 126 ± 12 |
| (rosette) [% (based on wild | | | | |
| type = 100%)] | | | | |

Results:

Overexpression of the pyrophosphatases in the transgenic Arabidopsis plants leads to a significant increase in the total shoot dry weight (rosette) of these plants in comparison with the wild-type Arabidopsis. In this connection, simultaneous overexpression of the two pyrophosphatases, i.e. both the cytosolic pyrophosphatase and the vacuolar pyrophosphatase, in Arabidopsis thaliana has a particularly marked effect on the total shoot dry weight; an increase of about 26% was achieved.

The transgenic plant which can be obtained in accordance with the invention exhibits an increased growth as the consequence of an increase in meristem activity.

### EXAMPLE 12

### Expression of V-PPase and C-PPase in Beta vulgaris

In order to investigate the effect of the overexpression of the Beta vulgaris cytosolic pyrophosphatase (C-PPase), of the overexpression of the Beta vulgaris vacuolar pyrophosphatase (V-PPase), or of the simultaneous overexpression of both pyrophosphatases on, mainly, the growth of the storage root, in particular the fresh beet weight, of Beta vulgaris, and also on the sucrose content in the beet body, transgenic Beta vulgaris beets were in each case prepared using the above-mentioned processes according to the invention. The vectors which were used for this purpose also contained the CaMV35S promoter in addition to the full-length cDNA of the respective pyrophosphatase. The respective pyrophosphatases were overexpressed under the control of this CaMV35S promoter. The effect on the Beta vulgaris fresh beet weight was examined in comparison with the wild-type Beta vulgaris 6 B 2840 (table 4).

TABLE 4

| | Wild | | | V-PPase & |
|---|---|---|---|---|
| | type | C-PPase | V-PPase | C-PPase |
| Beta vulgaris | 6 B 2840 | sense | sense | sense |
| Total fresh beet weight | 100 ± 12 | 112 ± 13 | 114 ± 11 | 119 ± 13 |
| [% (based on wild | | | | |
| type = 100%)] | | | | |

The effect on the sucrose content in the Beta vulgaris beet was examined in comparison with the sucrose content in the beet of the wild-type Beta vulgaris 6 B 2840 (table 5).

TABLE 5

| | | | | V-PPase & |
|---|---|---|---|---|
| | Wild type | C-PPase | V-PPase | C-PPase |
| Beta vulgaris | 6 B 2840 | sense | sense | sense |
| Sucrose content | 16 ± 2 | 18 ± 2 | 19 ± 3 | 21 ± 3 |
| [% by wt.] | | | | |
| Sucrose content | 100 | 112.5 | 118.75 | 131.25 |
| [% (based on wild | | | | |
| type = 100%)] | | | | |

Results:

The overexpression of the pyrophosphatases in the transgenic Beta vulgaris beets leads in each case to a significant increase in the fresh beet weight and the sucrose content of these plants as compared with the wild type. In this connection, the simultaneous overexpression of the two pyrophosphatases, i.e. both the cytosolic pyrophosphatase and the vacuolar pyrophosphatase, exerts a particularly marked effect on the fresh beet weight and sucrose content. An increase of about 19% was achieved in the case of the fresh beet weight. At the same time, the sucrose content was increased to a value of about 21%, which corresponded to an increase of about 31% as compared with the wild type.

The transgenic beet plants which can be obtained in accordance with the invention exhibit an increased sucrose content and an increased growth as the consequence of an increase in meristem activity.

1. A process for producing a transgenic sugar beet plant, which comprises:
a) transforming at least one sugar beet cell with at least two transgenes, with the first transgene encoding a vacuolar pyrophosphatase (V-PPase) and the second transgene encoding at least one of a cytosolic and a nucleus-located soluble pyrophosphatase (C-PPase),
b) culturing and regenerating the transformed cells under conditions which lead to the complete regeneration of the transgenic beet plant, and
c) obtaining a transgenic beet plant having at least one of an increased sucrose content in the beet, an increased meristem activity an extended meristem activity and a reduced rate of sucrose breakdown during storage.

2. The process as claimed in claim 1, wherein the first transgene comprises a nucleic acid sequence which is selected from the group of nucleotide sequences consisting of
a) a nucleotide sequence depicted in SEQ ID No. 4, or a sequence which is complementary thereto,
b) a nucleotide sequence encoding the amino acid sequence depicted in SEQ ID No. 5, or a sequence which is complementary thereto, and
c) a nucleotide sequence which exhibits a sequence identity of more than 80% with the sequence according to a) or b).

3. The process as claimed in claim 1, wherein the second transgene comprises a nucleic acid sequence which is selected from the group of nucleotide sequences consisting of
a) a nucleotide sequence depicted in SEQ ID No. 1, or a sequence which is complementary thereto,
b) a nucleotide sequence encoding the amino acid sequence depicted in SEQ ID No. 2, or a sequence which is complementary thereto, and
c) a nucleotide sequence which exhibits a sequence identity of more than 80% with the sequence according to a) or b).

4. The process as claimed in claim 1, wherein at least one of the first and the second transgene is arranged on a vector.

5. The process as claimed in claim 1, wherein the vector is equipped for overexpressing at least one of the first and the second transgene.

6. The process as claimed in claim 1, wherein at least one of the first and the second transgene is operatively linked, on the vector, to a promoter.

7. The process as claimed in claim 1, wherein the promoter is a tissue-specific promoter, a constitutive promoter, an inducible promoter or a combination thereof.

8. The process as claimed in claim 1, wherein the promoter is a promoter from Beta vulgaris, Arabidopsis thaliana or the cauliflower mosaic virus.

9. The process as claimed in claim 1, wherein the promoter is the CaMV35S promoter.

10. The process as claimed in claim 1, wherein the promoter is a Beta vulgaris V-PPase promoter.

11. The process as claimed in claim 10, wherein the promoter comprises a nucleotide sequence which is selected from the group of nucleotide sequences consisting of
a) a nucleotide sequence as depicted in SEQ ID No. 6 or 7, or a sequence which is complementary thereto, and
b) a nucleotide sequence which exhibits a sequence identity of more than 80% with one of the sequences as depicted in SEQ ID No. 6 or 7.

12. The process as claimed in claim 1, wherein the promoter is a sucrose synthase promoter.

13. The process as claimed in claim 1, wherein the promoter is a storage-specific promoter.

14. The process as claimed in claim 1, wherein the vector possesses intrans enhancers or other regulatory elements.

15. The process as claimed in claim 1, wherein the first and second transgenes are arranged together on a single vector.

16. The process as claimed in claim 1, wherein the first and second transgenes are arranged on different vectors.

17. The process as claimed in claim 1, wherein the first and second transgenes are transformed simultaneously.

18. The process as claimed in claim 1, wherein the transformation is at least one of a biolistic transformation, an electrotransformation, an agrobacterium-mediated transformation and a virus-mediated transformation.

19. A transgenic plant containing at least one transformed cell, said plant obtained using a process as claimed in claim 1.

20. The transgenic plant as claimed in claim 19, which exhibits an increased content of sucrose in comparison to a corresponding non-transgenic plant.

21. The transgenic plant as claimed in claim 19, which exhibits an increase in meristem activity during growth in comparison to a corresponding non-transgenic plant.

22. The transgenic plant as claimed in claim 19, which exhibits a decreased rate of sucrose breakdown during storage in comparison to a corresponding non-transgenic plant.

23. A harvesting or propagation material from a transgenic plant as claimed in claim 19.

24. A nucleic acid molecule encoding a protein having the biological activity of a Beta vulgaris soluble pyrophosphatase, with the sequence of the nucleic acid molecule being selected from the group of nucleotide sequences consisting of:
a) a nucleotide sequence depicted in SEQ ID No. 1, or a sequence which is complementary thereto,
b) a nucleotide sequence encoding the amino acid sequence depicted in SEQ ID No. 2, or a sequence which is complementary thereto, and
c) a nucleotide sequence which exhibits a sequence identity of more than 80% with the sequence according to a) or b).

25. A nucleic acid molecule encoding a promoter of a Beta vulgaris vacuolar pyrophosphatase (V-PPase), with the sequence of the nucleic acid molecule being selected from the group of nucleotide sequences consisting of
a) a nucleotide sequence as depicted in SEQ ID No. 6 or 7, or a sequence which is complementary thereto, and
b) a nucleotide sequence which exhibits a sequence identity of more than 80% with one of the sequences as depicted in SEQ ID No. 6 or 7.

26. A method for producing a transgenic plant which contains at least one transformed cell, said method comprising producing said plant with the use of the nucleic acid molecule as claimed in claim 24.

27. A vector which contains the sequence of the nucleic acid molecule as claimed in claim 24.

28. The vector as claimed in claim 27, which is a viral vector or a plasmid.

29. A method for producing a transgenic plant which contains at least one transformed cell, said method comprising producing said plant with the use of the vector claimed in claim 27.

30. A host cell which is transformed with a vector as claimed in claim 27.

31. The host cell as claimed in claim 30, which is a bacterial cell, plant cell or animal cell.


SEQUENCE LISTING (21 sequences)

SEQ ID NO: 1 - DNA, 1041 bp, Beta vulgaris
attataaaaacccctcaaaatcaggagaagtttaaggaatttgtagatctccgattcttctgtattcgttcattctaaaagctttcgattttacgctcttcgctaatttttctgaaacatggatgaggagatgaatgctgttgcggagatgaatgctgttgcttctaaagtaaaagaagagtatcgccgagctccgaagttgaaccaaaggatcatttcgtcaatgtcaaggagatctgttgcggcccatccttggcatgatctcgagattggacctaatgcccctgaaatctgtaactgtgttgttgagatacctaaagggagcaaggtcaagtatgagcttgacaagaaaactggacttattatggttgatcgaatattatactcatctgtggtctatcctcacaactatggttttattccaagaacattgtgcgaagatggtgaccccatggatgttttagtgctcatgcaggaaccagtcgtcccaggtcgctttcttcgagcccgggcaattggtttaatgcctatgattgatcagggggagaaagacgataagataattgcagtttgtgccgatgatcctgaagttcgccattacactgatatcaaccagcttcctcctcatcgtttggctgagatcagacgcttttttgaggactacaagaaaaatgagaacaaagaggttgcagtgaatgaatttttgccagctcaaattgctcatgatgccatccagcactctatggatctctatgcggaatacatcctacagacattgaggagatgatgaatggcactttcaattattgtcattcatatcctgaagtaatattgaaggcttttggtcacattgttacatcttatttttggtgctacctatttaagagtcgatgttggaaatcccaaaagaaagaaaaggagattttccctgttccttttctgaatcttcttgtcgaaaattttatgtattgtagtaaagctaaaacaatcttcatgaactttgaagttgagtttcctgtatcatgtttggtttaggaggct

SEQ ID NO: 2 - protein, 222 aa, Beta vulgaris
MDEEMNAVAEMNAVASKVKEEYRRAPKLNQRIISSMSRRSVAAHPWHDLEIGPNAPEICNCVVEIPKGSKVKYELDKKTGLIMVDRILYSSVVYPHNYGFIPRTLCEDGDPMDVLVLMQEPVVPGRFLRARAIGLMPMIDQGEKDDKIIAVCADDPEVRHYTDINQLPPHRLAEIRRFFEDYKKNENKEVAVNEPLPAQIAHDAIQHSMDLYAEYILQTLRR

SEQ ID NO: 3 - protein, 245 aa, Beta vulgaris
MRGSHHHHHHGSATMDEEMNAVAEMNAVASKVKEEYRRAPKLNQRIISSMSRRSVAAHPWHDLEIGPNAPEICNCVVEIPKGSKVKYELDKKTGLIMVDRIKYSSVVYPHNYGFIPRTLCEDGDPMDVLVLMQEPVVPGRFLRARAIGLMPMIDQGELDDKIIAVCADDPEVRHYTDINQLPPHRLAEIRRFFEDYKKNENKEVAVNEFLPAQIAHDAIQHSMDLYAEYILQTLRRVDLQPSLIS

SEQ ID NO: 4 - DNA, 2810 bp, Beta vulgaris
acactcttcctctccctctcttccaaaccctcttcattctctctctctctctctctctctctcctttatcttcttcttcttcttcaattttcttctcccattttcaaaaatcatgggtgcagctcttcttccagatctcataacagagattatcattcctgtatgtgctgtaattggaattgctttctctctctttcaatggtacatcgtttctcaggtcaagctttcccctgactctacccgcaataataacaacaaaaatggattttctgatagtttgattgaagaagaagaaggtcttaatgaccaaagtgttgttgctaaatgtgctgaaattcagaatgctatttctgaaggggcaacttccttccttttcaccgagtaccagtatgttggtatctttatggttgcttttgctgtgttgatattccttttcctcggatctgtggagggtttcagcacaagtagccaggaatgtacctatgacaaaaccaggaggtgcaagcctgctcttgccactgctatcttcagcacagtggccttcttgcttggcgctatcacttctttgggttctggtttcttcgggatgaagattgccacatacgcaaatgcccgaacaacactagaggctagaaagggtgtcggcaaagcattcattgtagcattcaggtctggagctgtcatgggattcctacttgctgcaaatggtcttttggtgctttacattactatccttctcttcaagatttactatggtgatgactgggaaggtctgtttgaggctataactggttatggtcttggaggatcatccatggcccttttcggtagagttgctggaggtatttacacaaaagctgccgatgtgggtgctgatcttgtcggtaaggttgaaagagacatccctgaggatgaccccagaaatccagctgttattgctgacaatgtcggcgacaatgttggggatatcgctggtatgggttctgatctttttggatcctacgctgagtcgtcctgtgctgctcttgttgttgcatccatttcctcattcgaaatttcccatgatttgacggcaatgatgtacccattgttggttagctcggttggtattattgtttgcttgatcacaaccttatttgcaaccgatttcttcgagatcaaggctgttaaggagattgagcctgcactcaagaagcagctaatcatctccactgctcttatgactgtcggagttgcagttatttcttggattgctcttcctacttcatttaccatttttgacttcggatctcagaaggaggtgcagaactggcaattgtttttatgtgttgctgttgggttgtgggctggctgtgcaagatgttgctgattcttgccgaactggagctgccacaaatgttatttttggcctggccttgggttacaaatcagtcattattcctatttttgccattgctatcagcattttcgtcagttttagctttgcagctatgtatggtattgctatggctgctcttggtatgctgagcaccattgccactggattggctattgatgcatatggccctatcagtgataatgctggaggcattgctgagatggctggtatgagccacagaatccgtgagagaactgatgcccttgatgctgctggaaacacaaccgctgctattggaaagggttttgcaatcggttctgcagctcttgtttctcttgctctctttggtgcttttgtaagccgtgcatccatccaaactgtggatgtgttgaccccgaaagtattcattggtctcattgtgggagccatgcttccatactggttctctgccatgacaatgaagagtgtgggaagtgcagctttgaaaatggttgaggaggtccgaaggcaattcaacaccatccctggcttgctggaaggcactgccaaacccgactatgctacctgtgtcaagatctccactgatgcttccatcaaggagatgatccccccaggtgctcttgtcatgctcacaccattgattgttggaaccttctttggtgtcgaaactctgtctggcgttcttgctggttctcttgtgtctggtgtacagattgctatttctgcatccaacactggtggtgcttgggacaatgccaagaagtacattgaggctggtgcttcagagcatgcaaggacacttggtcccaagggatcagatgcacacaaggcagctgtgatcggtgacaccatcggtgacccacttaaggacacatcaggaccatcactcaacattctaatcaagcttatggctgtcgagtcactagtgttcgcccccttcttcgccacccacggtggcttgctcttcaagtacctctaaatatgatcggcgcaaaatcagaaggcgacagagggaggaattcgcggtttcttctcctcattttgtcgcctacaaatcgggcaagttttaaattttatcgcacaatttttgaatgtcgttagatgacaactacaaggctggaggggctaaaacttctacatgatgatgatgataatgataatttggaagcaagtcttgtgaaaaatagagttatatggtcaacattattcttttcttttttcttccttttattgtaagatcgggatttgtagtaatcattttgcaaacctcttttgttaggtataactcattttctattttagtccttcagaaattgcatgcagttgcccttttattttctaaaaagagaacctgttcttgagcatgtgttgtaagggcagaatgttctcatgtactttcttggaatttatctcattttgcagattggatcta

SEQ ID NO: 5 - protein, 764 aa, Beta vulgaris
MGAALLPDLITEIIIPVCAVIGIAFSLFQWYIVSQVKLSPDSTRNNNNKNGFSDSLIEEEEGLNNQSVVAKCAEIQNAISEGATSFLFTEYQYVGIFMVAFAVLIFLFLGSVQGFSTSSQECTYDKTRRCKPALATAIFSTVAFLLGAITSLGSGFFGMKIATYANARTTLEARKGVGKAFIVAFRSGAVMGFLLAANGLLVLYITILLFKITTGDDWQGLFQAITGYGLGGSSMALFGRVAGGIYTKAADVGADLVGKVERDIPEDDPRNPAVIADNVGDNVGDIAGMGSDLFGSYAESSCAALVVASISSFEISHDLTAMMYPLLVSSVGIIVCLITTLFATDFFEIKAVKEIEPALKKQLIISTALMTVGVAVISWIALPTSFTIFDFGSQKEVQNWQLFLCVAVGLWAGLIIGFVTQYYTSNAYSPVQDVADSCRTGAATNVIFGLALGYKSVIIPIFAIAISIFVSFSFAAMYGIAMAALGMLSTIATGLAIDAYGPISDNAGGIAEMAGMSHRIRERTDALDAAGNTTAAIGKGFAIGSAALVSLALFGAFVSRASIQTVDVLTPKVFIGLIVGAMLPYWFSAMTMKSVGSAALKMVEEVRRQFNTIPGLLQGTAKPDYATCVKISTDASIKEMIPPGALVMLTPLIVGTFFGVQTLSGVLAGSLVSGVQIAISASNTGGAWDNAKKTIEAGASEHARTLGPKGSDAHKAAVIGDTIGDPLKDTSGPSLNILIKLMAVESLVFAPFFATHGGLLFKYL

SEQ ID NO: 6 - DNA, 1733 bp, Beta vulgaris
atcctccatcgattcacataggatgtgaaccgttgatttttttttttttttaaaaagttcagtgcaaaagttagaaattacttaaggcaaatcgctatttcaattagcgattttattaaaatagatcactaactgaagcctgtttactatcattttttgtttttagctttcaaaatttctaaaaactataaacaagatgataaaaaccacaaaaaatagttttaagttattagttttcaaaattgagaagactatatattatagcaatgaatacttttaagtttattatactgtttatatcatatgacttttaaaaccatcaaccaaaaattgaaaattaatagtgatgttgaacaaccctaagttagcattttctattttacaaaaccactaactcggatagcgatttaattaagttaaaccactaactcaaaattagcggtttaattcgggtacatcacaaaccattcacataacacttgaacaatattttctaaaataaaaactaacctaaaccgctaactcaattagtgatgttgagagtatttttgtccttctttaacctcacagctaatggttttgttcattataagtgtcacttcaataaaatgattctcatagttatctttaaaaagtgttcttttatgttaaaaacaattaagttcaatgacataaacgagattcgatcccacacaagactttaccagttaagctatataacatccatcagtatctaaaaagaagtcggtacctgacaatgacggtaaaaaagcaccttaaaaaagtaatactatgtgaatttaggttccttatcaagcgcttcagaaacacctattatcaatcaaagaaataatagtaataataataataccaataaaaataattaaaatgaattacaaaatataatactccacctaattataatttactagaattttttgcacgcgatgcgtgcttgaatttttttcgaaaaagaaactcgatttttttcgacataagagtcaaaatttgaacattagacaaacgaagtataattatttttagttgcaaaatttgattggcttagtttctatcacttatatctctcaccattcttttttttttttatacttttcaaagttaaattatatgaacaaaagagaaattttattgaatttatttataatttttaatattataattttttagttgatttttgaattaagtacagtactttataaattgtaaagaaagtgtacactttgatttcaagtcaattttttcataggttgtagtttgtaagtgaatttttttgtttttgtaaagtttattcattttagtgatttgcataacgtaaattatgcaattttatgattttagttgacttgtgagtgattgttataattatatttttggcatttttgtttgaagcccactttaatttgtaagtgaatttgttatttagaatgagaagggggtaaaatagacatttcaaaataggacaccattgctcccctttcccttatataatagagataagtagtaaataaatagaaagtaaaaacccctcaactttgaggagtacttaccttaattaatatcccatttcccttgtcaatcctccctataaaacaaaacccacacttctcacactcttcctctccctctcttccaaaccctcctcattttctctctctctctcctttatcttcttcttcttcttcaattttcttctcccattttcaaaaatcatg

SEQ ID NO: 7 - DNA, 962 bp, Beta vulgaris
tcgaatttacaataatttattttgcacataaaaattgacgttgttgcgcataaattgaatataatataaaagattattgactacatcacataaaatctgattatgagtgagttctttcttcacctaaataacatgaacttatttaaactgacttattaaaccttattgatccttacttgaacgtatatttgagtattatctcagacctgatcaattataatcagactatatctgaacttattagacctaaaatttattttttaagttgaagagaatataccttataattcatattaaaaaattaactacatatacaaaaaatgattatttaaaaaataattatatcaaataaaaacggactatattatactaaagctatatttagttcacccgaattttttgattagaacttatgttttctaatctgatctgatctgaactgatctgattacaagatcttatcttttagatttttctcaatatataagaaaaaatataatcatgtggggtcttgtttgattcgtatcaatgagtactttattcatgttcaattattataatttttactaatacgtgaagaaagatatttaatagtaataatgatttttaaatatgagcatgatctgaactgatttgatctgaactttttttttatctgatctgaaataagtaaaaataagctcaactaaacatggcctaagtataattttcaataaacaacattaagttattatgaatgtaatccatttcaagttttttttaaaaccctattacacctcaccacacccaataaaaacccgtcctaatttctcctcactataaaactaaaaacccactccactctcttacacacactccacactcaaattgtgttgttgtcttaactgtattttctctgttgccggaatttcggcgatttttagggttccggcgtaaagttagggttccggtgaagaaaaatg

SEQ ID NO: 8 - DNA, 18 bp, Beta vulgaris
tgctgctcatccwtggca

SEQ ID NO: 9 - DNA, 22 bp, Beta vulgaris
tcrttyttcttgtartcytcaa

SEQ ID NO: 10 - DNA, 38 bp, Beta vulgaris
gtcgggatccgccaccatggatgaggagatgaatgctg

SEQ ID NO: 11 - DNA, 38 bp, Beta vulgaris
gaagctgcaggtcgactctcctcaatgtctgtaggatg

SEQ ID NO: 12 - DNA, 31 bp, Beta vulgaris
ccggggtaccaaggaatttgtagatctccga

SEQ ID NO: 13 - DNA, 31 bp, Beta vulgaris
ctagtctagaagcctcctaaaccaaacatga

SEQ ID NO: 14 - DNA, 30 bp, Beta vulgaris
acactcttcctctccctctcttccaaaccc

SEQ ID NO: 15 - DNA, 31 bp, Beta vulgaris
tagatccaatctgcaaaatgagataaattcc

SEQ ID NO: 16 - DNA, 34 bp, Beta vulgaris
aagtcggggcccgaattcccatggagtcaaagat

SEQ ID NO: 17 - DNA, 35 bp, Beta vulgaris
gaagccatcgataagcttggacaatcagtaaattg

SEQ ID NO: 18 - DNA, 20 bp, Beta vulgaris
ggwgghattgctgaratggc

SEQ ID NO: 19 - DNA, 21 bp, Beta vulgaris
agtayttcttdgcrttvtccc

SEQ ID NO: 20 - DNA, 24 bp, Beta vulgaris
ccaaaacgtcgtcgctaaatgtgc

SEQ ID NO: 21 - DNA, 20 bp, Beta vulgaris
accggaaccctaactttacg